(19) United States
(12) Patent Application Publication
(10) Pub. No.: US 2010/0035982 A1
(43) Pub. Date: Feb. 11, 2010

(54) OXALIPLATIN FORMULATIONS

(75) Inventors: Darryl Vanstone Whittaker, Vermont (AU); Aikun Julie Liu, Endeavour Hills (AU)

Correspondence Address: WINSTON & STRAWN LLP;PATENT DEPARTMENT, 1700 K STREET, N.W., WASHINGTON, DC, 20006, US

(73) Assignee: Mayne Pharma Limited

(21) Appl. No.: 12/581,624

(22) Filed: Oct. 19, 2009

Related U.S. Application Data
(63) Continuation of application No. 11/627,648, filed on Jan. 26, 2007.
Application No. 11/627,648 is a continuation of application No. 10/927,279, filed on Aug. 27, 2004, now abandoned.

(30) Foreign Application Priority Data
Aug. 28, 2003 (AU) 2003904627

(51) Int. Cl. A61K 31/282; A61P 35/00

(52) U.S. Cl. 514/492

(57) ABSTRACT

The present invention provides a pharmaceutical liquid formulation of oxaliplatin for parenteral administration, said formulation having (i) oxaliplatin, (ii) water; and (iii) an additive selected from the group of tartaric acid, a salt of tartaric acid, a pharmaceutically acceptable derivative of tartaric acid and mixtures thereof.

### CROSS-REFERENCE TO RELATED APPLICATIONS

This application is a continuation of U.S. patent application Ser. No. 11/627,648, filed Jan. 26, 2007, which is a continuation of U.S. patent application Ser. No. 10/927,279, filed Aug. 27, 2004, now abandoned, which claims priority to Australian Patent Application No. 2003904627, filed Aug. 28, 2003, the entire disclosure of each of which is incorporated herein by reference.

### FIELD OF THE INVENTION

The invention relates to formulations containing oxaliplatin and tartaric acid.

### BACKGROUND OF THE INVENTION

Oxaliplatin is an anticancer agent. Oxaliplatin (CAS 61825-94-3), also known as L-OHP, is a third generation platinum complex. The term “oxaliplatin” as used herein includes cis-oxalato(trans-l-1,2-diaminocyclohexane) platinum(II), its optic enantiomer cis-oxalato(trans-d-1,2-diaminocyclohexane) platinum(II), and any mixture thereof.

Oxaliplatin is currently approved and marketed for second-line treatment of colorectal cancer. Oxaliplatin is available in a lyophilised form (20 mg, 50 mg or 100 mg vials). Just prior to administration, the lyophilised powder is reconstituted using water for injection or 5% glucose injection solution to provide a solution containing 5 mg/ml oxaliplatin. Typically, the reconstituted solution is then further diluted in 250-500 mL of 5% glucose injection solution. The diluted oxaliplatin solution is then infused either by peripheral vein or central venous line over 2 to 6 hours.

Lyophilized oxaliplatin has some disadvantages as a pharmaceutical form. The manufacturing process for a lyophilised dosage form is complicated and expensive. For example, the risk of sterility failure during manufacture of freeze dried forms is generally higher than is the case for liquid solutions. In addition, the reconstitution of freeze dried preparations requires both skill and care as it involves several risks, inter alia, incomplete dissolution of the powder, contamination through handling a highly toxic substance as a powder or cake, and maintaining the sterility of both the vial and the infusion solution during reconstitution and transfer to the infusion bag. Thus, to administer a lyophilized drug, multiple handling of the drug is required—the lyophilised oxaliplatin is first reconstituted, then diluted with a 5% glucose solution and then administered by intravenous infusion.

Further, following reconstitution, oxaliplatin is prone to instability, particularly in solutions containing certain nucleophilic agents. For example, some reconstitution solutions containing chloride ions, such as 0.9% sodium chloride solution, are commonly used in hospitals. The mistaken use of such a reconstitution solution in the case of the lyophilized form of oxaliplatin has the serious consequence of rapidly decomposing the oxaliplatinum metal complex, forming a precipitate (dichloro-diaminocyclohexane-platinum derivative) with NaCl.

As a consequence of the limitations described above, several stabilised aqueous ready-to-use (RTU) liquid oxaliplatin preparations have been proposed:
1. U.S. Pat. No. 5,716,988 and AU 731981 disclose a pharmaceutical formulation consisting of a 1 to 5 mg/mL solution of oxaliplatin in water having a pH range of 4.5 to 6. However, subsequently, WO 99/43355 and U.S. Pat. No. 6,476,068 report that simple aqueous solutions of oxaliplatin prepared according to the methods taught in this specification are insufficiently stable.
2. WO 99/43355 and U.S. Pat. No. 6,306,902 disclose an oxaliplatin solution formulation containing 1 to 7 mg/ml oxaliplatin, a buffering agent and a pharmaceutically acceptable carrier. The preferred buffering agent (and only example) is oxalic acid or an alkali metal salt thereof.
3. WO 01/15691 discloses solutions of at least 7 mg/ml oxaliplatin containing a solvent containing a sufficient amount of at least one hydroxylated derivative selected from 1,2-propane-diol, glycerol, maltitol, sucrose and inositol. The specification states that these are the only suitable agents to use after consideration of several options. Further, if buffering agents are used, the specification teaches that the buffer should have an oxalic acid base.
4. U.S. Pat. No. 6,476,068 discloses an oxaliplatin solution formulation comprising 0.1 to 10 mg/ml oxaliplatin, an effective stabilizing amount of the monocarboxylic acid, lactic acid, and a pharmaceutically acceptable carrier. The preferred concentration range of oxaliplatin is 2 to 5 mg/ml.
5. US Patent Application No. 20030109515 discloses an oxaliplatin solution formulation containing a stabilising amount of malonic acid. The examples are directed to formulations having an oxaliplatin concentration of 2 mg/ml. In contrast to the teaching of this application, and as is discussed below, the present inventors have found that malonic acid destabilises oxaliplatin in solution.

Buffering agents are used in liquid pharmaceutical formulations to adjust the pH of the formulation and to maintain the formulation within a desired pH range. As mentioned above, the dicarboxylic acid, oxalic acid, and its salts have been proposed as a buffering and stabilising agent for oxaliplatin. Oxalate ion is formed in aqueous solutions of oxaliplatin by hydrolysis, thus conceivably this reaction may be slowed (using Le Chatelier's principle) through purposeful addition of oxalate ion to solutions of oxaliplatin. However, oxalic acid has some disadvantages as a pharmaceutical buffering agent, notably it's toxicity. Oxalic acid is potentially nephrotoxic and also requires special handling precautions, which complicate and limit its use in pharmaceutical products.

As is known to a person skilled in the art a buffering system is a mixture of an acid with it conjugate base in a solution, the mixture being formulated so as to maintain the pH of the solution at a desired level. As defined herein, “buffering agent” refers to an acid or a base which may form a component of a buffering system whether or not the acid or base is associated with its conjugate base or conjugate acid, respectively.

There is a need for buffering agents for oxaliplatin solutions that can be used as alternatives to the prior art buffering agents (oxalic acid, lactic acid and malonic acid) and which do not have the disadvantages associated with the use of oxalic acid.

Ideally, the alternative buffering agents would not destabilise oxaliplatin in solution. In particular, it would be useful if the alternative buffering agents improve the stability of oxaliplatin in aqueous formulations in a manner that minimises significant degradation of oxaliplatin and limits the formation of unwanted impurities such as diaquo DACH platinum and diaquo DACH platinum dimer.

Further, it would be preferable to limit the amount of unknown degradation products in the aqueous formulation. Any unknown degradation product present in an amount exceeding the thresholds set in the guidelines of the ICH (International Conference on Harmonisation of Technical Requirements for Registration of Pharmaceuticals for Human Use) is required to be identified. This imposes significant requirements on the manufacturer of the formulation, as they are required to identify trace amounts of an unknown degradation product. In addition, the presence of unknown degradation products is an indication that there may be additional risks of toxicity and unknown side-effects as a consequence of the presence of these products. It is therefore of interest to a manufacturer of a formulation to avoid producing unknown degradation products.

Ideally, additional pharmaceutically acceptable buffering agents should be non-toxic and be present in the smallest possible quantity. Furthermore, during manufacture they should be introduced in the safest and most convenient manner possible.

### SUMMARY OF THE INVENTION

In a first aspect the present invention provides a pharmaceutical liquid formulation of oxaliplatin for parenteral administration, said formulation comprising
(i) oxaliplatin,
(ii) water; and
(iii) an additive selected from the group consisting of tartaric acid, a salt of tartaric acid, a pharmaceutically acceptable derivative of tartaric acid wherein said pharmaceutically acceptable derivative is selected from the group consisting of esters, amides, carbonates and carbamates of tartaric acid, and mixtures thereof.

In a second aspect, the present invention provides a method for treating a cancer which comprises administering a pharmaceutical formulation according to the first aspect of the invention to a patient in need thereof.

In a third aspect, there is provided a method for preparing pharmaceutical formulations according to the first aspect, the method comprising the steps of:
(i) dissolving oxaliplatin in water to form a solution;
(ii) dissolving the additive in the solution; and
(iii) optionally, adjusting the pH of the solution with a pharmaceutically acceptable base to form the pharmaceutical formulation.

In a fourth aspect, the present invention provides a pharmaceutical liquid formulation of oxaliplatin for parenteral administration, said formulation comprising
(i) about 5 mg/ml of oxaliplatin,
(ii) water, and
(iii) an additive consisting of tartaric acid and the sodium salt of tartaric acid, wherein the concentration of the additive is about 0.2 mM and wherein the pH of the solution is from 4.7 to 5.5.

In a fifth aspect, the present invention provides a method for treating a cancer which comprises administering a pharmaceutical formulation according to the seventh aspect to a patient in need thereof.

In a sixth aspect, there is provided a method for preparing a pharmaceutical formulation, the method comprising the steps of:
(i) dissolving oxaliplatin in water to form a solution;
(ii) dissolving tartaric acid in the solution;
(iii) adjusting the pH of the solution with sodium hydroxide such that it is in the range of from 4.7 to 5.5 to form the pharmaceutical formulation
wherein the concentration of oxaliplatin is about 5 mg/ml and the concentration of tartaric acid is about 0.2 mM.

### BRIEF DESCRIPTION OF THE FIGURES

FIG. 1(a) is a chromatogram showing stability of a solution of oxaliplatin in water stored at 40° C. for 12 weeks.

FIG. 1(b) is a chromatogram showing stability of a solution of oxaliplatin and tartaric acid in water stored at 40° C. for 12 weeks.

FIG. 1(c) is a chromatogram showing stability of a solution of oxaliplatin, tartaric acid and sodium tartrate in water stored at 40° C. for 12 weeks.

FIG. 2(a) is a chromatogram showing stability of a solution of oxaliplatin in water stored at 40° C. for 8 weeks.

FIG. 2(b) is a chromatogram showing stability of a solution of oxaliplatin and tartaric acid in water stored at 40° C. for 8 weeks.

FIG. 2(c) is a chromatogram showing stability of a solution of oxaliplatin, tartaric acid and sodium tartrate in water stored at 40° C. for 8 weeks.

FIG. 2(d) is a chromatogram showing stability of a solution of oxaliplatin, tartaric acid and sodium tartrate in water at 40° C. for 8 weeks, the ratio of tartrate to tartaric acid being greater than for the solution of FIG. 2(c).

### DETAILED DESCRIPTION OF THE INVENTION

In a first aspect, the present invention provides a pharmaceutical liquid formulation of oxaliplatin for parenteral administration, said formulation comprising:
(i) oxaliplatin,
(ii) water; and
(iii) an additive selected from the group consisting of tartaric acid, a salt of tartaric acid, a pharmaceutically acceptable derivative of tartaric acid and mixtures thereof;
   wherein the additive is at a concentration of at least 0.01 mM.

As is well known tartaric acid is found in various isomeric forms. The present invention contemplates the use of any of the isomers of tartaric acid as an additive. For instance, the tartaric acid may be selected from any of the isomers of tartaric acid including the group consisting of (+)-tartaric acid, (−)-tartaric acid, mesotartaric acid and mixtures thereof. Preferably, the tartaric acid is (+)-tartaric acid.

Where the additive is a mixture of a tartaric acid and tartrate the concentration of the additive is the sum of the concentrations of the tartaric acid and the tartrate.

When the additive includes a salt of tartaric acid, the salt may be formed in situ by the addition of a pharmaceutically acceptable base to an acid solution. Alternatively, the salt may be added directly to the formulation.

Preferably, the concentration of the additive is from about 0.01 mM to about 2.0 mM, more preferably from about 0.1 mM to about 1.0 mM, even more preferably from about 0.1 mM to about 0.6 mM, yet more preferably from about 0.2 mM to about 0.6 mM.

When the additive comprises a salt of tartaric acid the salt is preferably a sodium salt.

Pharmaceutically acceptable derivatives of tartaric acid include but are not limited to derivatives such as esters, amides, carbonates and carbamates of the acid.

The amount of oxaliplatin present in a pharmaceutical formulation according to the invention is preferably up to about 15 mg/ml, preferably about up to about 7 mg/ml. Preferably the amount of oxaliplatin is in the range of from 1 to 5 mg/ml and most preferably is about 5 mg/ml.

As will be understood, the additive should be used at a concentration which does not destabilise the oxaliplatin and preferably aids stability of the oxaliplatin. The desired stability of oxaliplatin will depend on the intended shelf life of the pharmaceutical formulation and the manipulation prior to administration. More specifically, a stable aqueous oxaliplatin formulation is one in which there will be no significant change in oxaliplatin potency at the specified storage condition. The criteria for “significant change” are as defined in the International Conference on Harmonisation (ICH) Guideline: Stability Testing of New Drug Substances and Products Q1A (R2). Thus in the case of injectable RTU oxaliplatin solution, potency of oxaliplatin should be at least 95% of initial content, and solution remains clear, colourless and free of precipitation for a pharmaceutically acceptable duration of time.

Preferably, the additive is at a concentration sufficient to buffer the formulation at a pH in the range of from about 3 to about 8, more preferably about 3 to about 7, even more preferably about 5.

Preferably the pharmaceutical formulation of the invention is provided in a sterile, sealed container. For example, a neutral glass of type I and a stopper. Examples of the stopper include those made of an elastomer based on halogenated butyls, possibly coated with a fluorinated polymer.

In a second aspect of the present invention there is provided a method for treating a cancer which comprises administering a pharmaceutical formulation according to the first aspect of the invention to a patient in need thereof.

The cancer can be any cancer that is amenable to treatment by oxaliplatin, either alone or in combination with other chemotherapeutic agents, and includes colorectal cancer.

The term “treating” as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

In the above methods, the effective dosage of oxaliplatin to be administered to a patient ranges from about 10 mg/m2 to about 250 mg/m2, more preferably from about 30 mg/m2 to about 180 mg/m2 and most preferably is about 85 mg/m2. However, it will be understood that the therapeutic dosage administered will be determined by the physician in the light of the relevant circumstances including the severity of the condition to be treated and the chosen route of administration. Therefore, the above dosage ranges are not intended to limit the scope of the invention in any way. Administration of oxaliplatin will typically be according to best practice known to those skilled in the art at the time of administration.

The present invention yet further provides a method for preparing a pharmaceutical formulation, the method comprising the steps of:
(i) dissolving oxaliplatin in water to form a solution;
(ii) dissolving in the solution an additive selected from the group consisting of a tartaric acid, a salt of tartaric acid, a pharmaceutically acceptable derivative of a pharmaceutically acceptable tartaric acid and mixtures thereof;
(iii) optionally, adjusting the pH of the solution with a pharmaceutically acceptable base.
   pH adjustment may be carried out with any pharmaceutically acceptable base. Preferably the pharmaceutically acceptable base is a sodium hydroxide (NaOH) solution.

In a further aspect, the present invention provides a pharmaceutical liquid formulation of oxaliplatin for parenteral administration, said formulation comprising
(i) about 5 mg/ml of oxaliplatin,
(ii) water, and
(iii) an additive consisting of tartaric acid and the sodium salt of tartaric acid,
   wherein the concentration of the additive is about 0.2 mM and wherein the pH of the solution is from about 4.7 to about 5.5.

In a still further aspect, the present invention provides a method for preparing a pharmaceutical formulation, the method comprising the steps of:
(i) dissolving oxaliplatin in water to form a solution;
(ii) dissolving tartaric acid in the solution;
(iii) adjusting the pH of the solution with sodium hydroxide such that it is in the range of from 4.7 to 5.3
   wherein the concentration of oxaliplatin is about 5 mg/ml and the concentration of tartaric acid is about 0.2 mM.

In order that the nature of the present invention may be more clearly understood, preferred forms thereof will now be described with reference to the following non-limiting examples.

### EXPERIMENTAL

### Measurement of Stability of Oxaliplatin Formulations

The stability of an oxaliplatin formulation over a period of time can be measured by a number of complementary methods. Visual appearance and stability of the pH of the formulation are important indicators and these can be measured by techniques well known to those skilled in the art.

Stability can also be measured by high pressure liquid chromatography (HPLC) techniques. HPLC is a technique that is widely used and well known in the art. HPLC can be used to measure the potency of the oxaliplatin where potency is defined as a percentage of the initial concentration of oxaliplatin. HPLC can also be used to measure the relative proportions of known and unknown degradants in an oxaliplatin solution.

Known degradation products of oxaliplatin include:
(trans-l-1,2diaminocyclohexane)trans-dihydroxo(oxalato) platinum (IV). This a oxidative degradation product of oxaliplatin. This degradation product has been designated as Impurity C in the Examples.
(SP-4-2)-diaqua-[(1R,2R)-cyclohexane-1,2-diamine-kN,kN′]platinum, or diaqua DACH platinum. This is a hydrolysis degradation product of oxaliplatin. This degradation product has been designated as Impurity B in the Examples.
(SP-4-2)-di-μ-oxobis[(1R,2R)-cyclohexane-1,2-diamine-kN,kN′]platinum, or diaqua DACH platinum dimer. This is a degradation product resulting from further reaction of Impurity B. This degradation product has been designated as Dimer in the Examples.
   R,S-oxaliplatin is an isomeric form of oxaliplatin which is found at low levels as an impurity in oxaliplatin (ie cis-oxalato(trans-l-1,2-diaminocyclohexane) platinum(II)).

### Overview of the Examples

Example 1 details an initial trial of oxaliplatin formulations containing tartaric acid over a pH range from 3 to 7 in and their ability to stabilise oxaliplatin was compared to a control. Tartaric acid was found to stabilise oxaliplatin and it was subsequently tested across a wide pH and concentration range as reported in Example 2. This study confirmed the advantages of tartaric acid and also indicated that there was a preferred concentration range for improved stability. Example 3 provides details of an aqueous solution of oxaliplatin and tartaric acid which was prepared for regulatory testing.

### Example 1

The stability of an array of oxaliplatin formulations containing tartaric acid in water for injection (WFI) having an oxaliplatin concentration of 5 mg/ml was assessed. The pH of the formulations covered a range of values.

### Comparative Example 1(a)

### Preparation of the Control Solution

WFI (water for injection) was added to a suitable glass vessel to about 80% of the desired quantity of final volume and warmed to 45-50° C. While stirring and flushing with nitrogen, the desired quantity of oxaliplatin (calculated at 5 mg/mL at the final desired volume) was added and dissolved. The solution was then made up to the desired final volume with WFI.

### Example 1(b)

### Preparation of Tartaric Acid Solutions

For the formulations described below, WFI was added to a suitable glass vessel to about 80% of the desired final volume and warmed to 45-50° C. While stirring and flushing with nitrogen, the desired quantity of oxaliplatin was added and dissolved. Thereafter the proposed stabilising dicarboxylic acid or its alkali salt was added to the oxaliplatin solution until completely dissolved. Where required, pH was adjusted through the addition of dilute NaOH solution. The solution so formed was made up to the final volume with WFI.

TABLE 1
Oxaliplatin formulations containing tartaric acid based agents

| | Tartaric | | |
|---|---|---|---|
| Formulation | pH 3 | Tartaric pH 5 | Tartaric pH 7 |
| Oxaliplatin | 5 mg | 5 mg | 5 mg |
| NaOH 10N | N/a | 0.033 μL | 0.067 μL |
| Initial pH of the final | 3.56 | 4.8 | 7.08 |
| formulation | | | |
| Molarity of Tartaric | 2.9 × 10⁻⁴ M | 2.2 × 10⁻⁴ M | 3.1 × 10⁻⁴ M |
| Acid/Tartrate in Solution | | | |

The pH values used to designate the different formulations are indications only and do not necessarily reflect the exact pH of each solution. The exact initial pH values are provided in the tables above.

### Example 1(c)

### Stability Study

In accordance with an accelerated stability protocol, the formulations were stored at 40° C. with 75% relative humidity for 12 weeks.

The potency of the formulations was examined by high performance liquid chromatography (HPLC) at 4 week intervals over the 12 week period. Potency is defined as a percentage of the initial concentration of oxaliplatin. The formulations maintained at least 95% potency over the 12 week period.

Only very low levels of the oxidative degradation product Impurity C [(trans-1-1,2-diaminocyclohexane)trans-dihydroxo(oxalato) platinum (IV)] were detected in the formulations. This indicated that the formulations were substantially free of oxygen.

### Example 1(d)

### Study of the Degradation Products of Oxaliplatin at 12 Weeks

Formulations Control, Tartaric pH 3 and Tartaric pH 7 were analysed after 12 weeks at 40° C. with 75% relative humidity for the presence of major degradation products of oxaliplatin [Impurity B(diaqua DACH platinum) and Dimer (diaqua DACH dimer)] using HPLC.

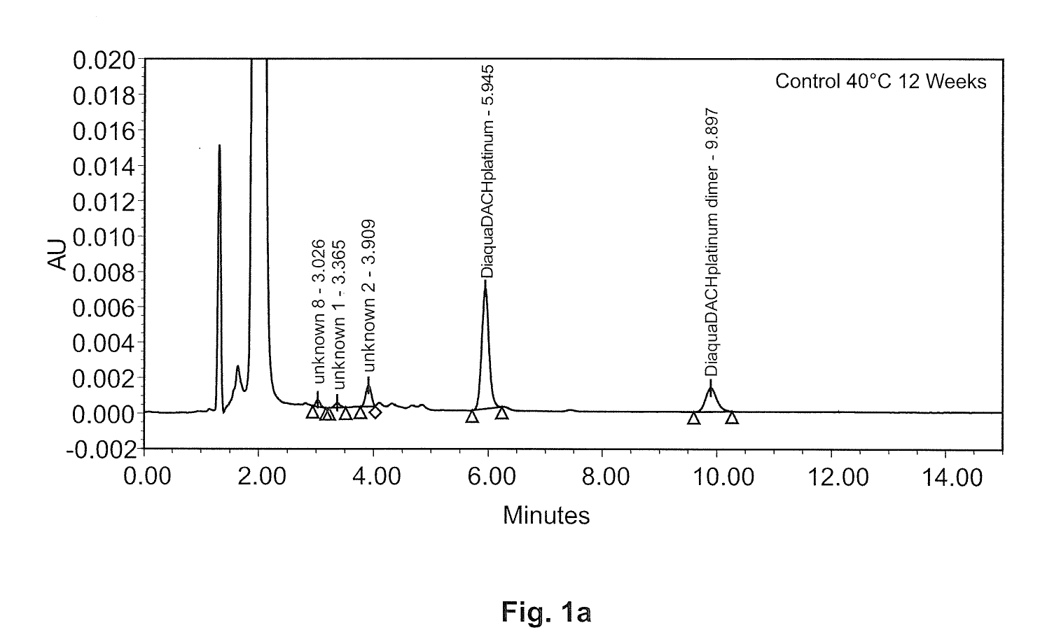

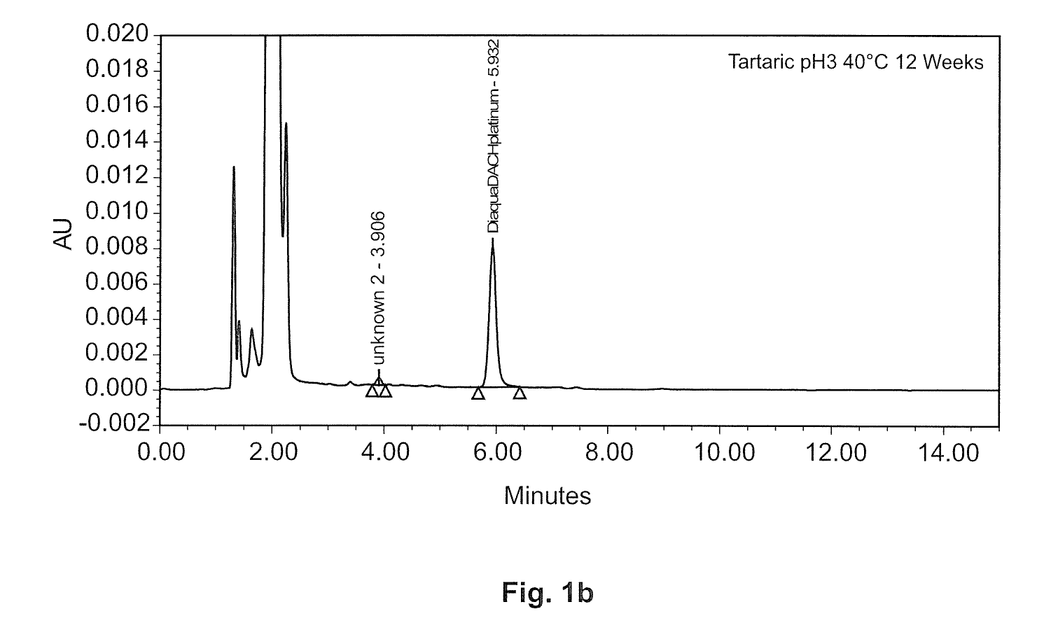

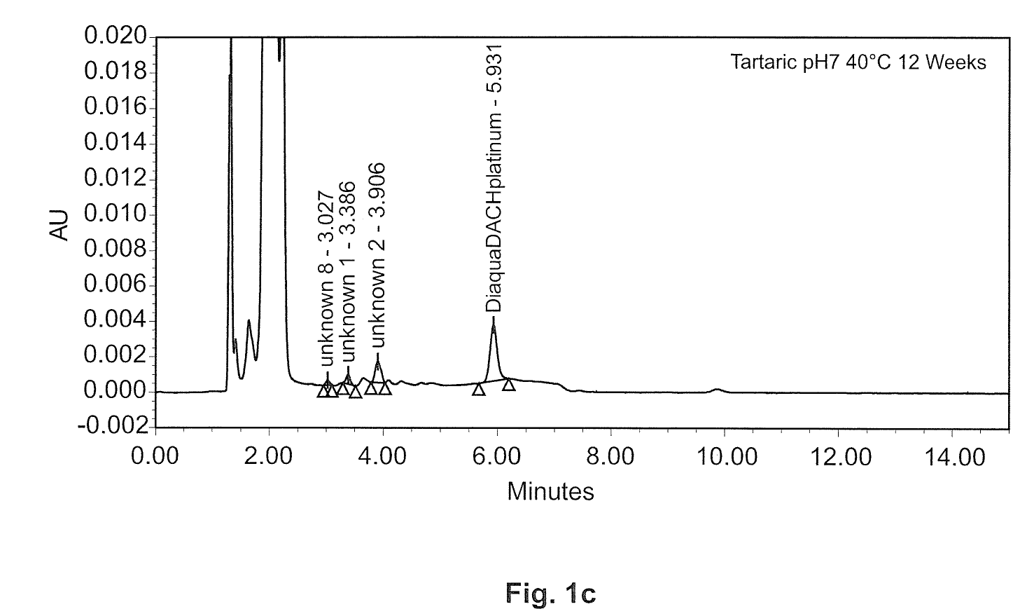

The chromatograms of the formulations are presented in FIG. 1(a)-(c). The impurity peaks at above 0.01% are reported.

FIG. 1(a)

### Control 40° C. 12 Weeks

This system displays an impurity peak at 5.945 minutes corresponding to Impurity B (diaqua DACH platinum) and a further peak at 9.897 minutes corresponding to Dimer (diaqua DACH platinum dimer). A further three unknown impurity peaks are present. One is present at 3.909 minutes at a level of 0.03% and two at 3.026 and 3.386 minutes at 0.01%.

FIG. 1(b)

### Tartaric pH3 40° C. 12 Weeks

An impurity peak is present at 5.932 minutes which has been allocated to impurity B (diaqua DACH platinum). There is also present an impurity at 3.906 minutes. There is no impurity peak corresponding to Dimer (diaqua DACH platinum dimer).

FIG. 1(c)

### Tartaric pH7 40° C. 12 Weeks

This system displays an impurity peak is present at 5.931 minutes which corresponds to Impurity B (diaqua DACH platinum). There are also three unknown impurity peaks eluted at 3.027 minutes, 3.387 minutes and 3.906 minutes at the level of 0.01, 0.01 and 0.03% respectively. There is no impurity peak corresponding to Dimer (diaqua DACH platinum dimer).

In comparison to the chromatogram of the control formulation, the formation of Dimer (diaqua DACH platinum dimer) is suppressed in the tartaric acid formulations. Further, at least in the case of the Tartaric pH 7 formulation, significantly less Impurity B (diaqua DACH platinum), the principle degradant, is formed. In addition, the tartaric acid formulations do not display as many unknown impurity peaks as the control formulation.

### Example 1(e)

### Study of the Degradation Products of Oxaliplatin at 8 Weeks

Formulations Control, Tartaric pH 3, Tartaric pH 5 and Tartaric pH 7 were analysed after 8 weeks at 40° C. with 75% relative humidity for the presence of degradation products of oxaliplatin using HPLC.

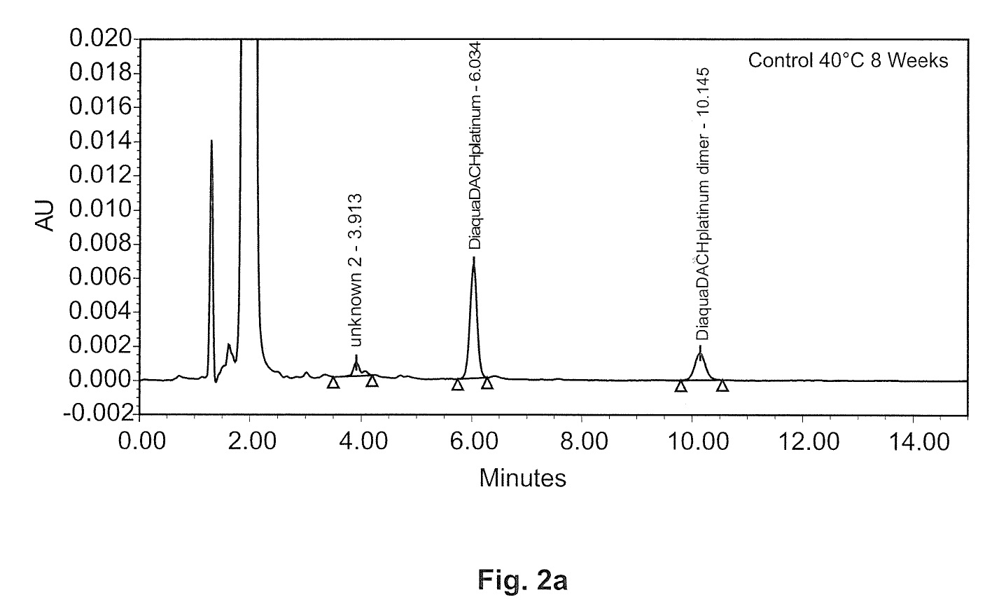

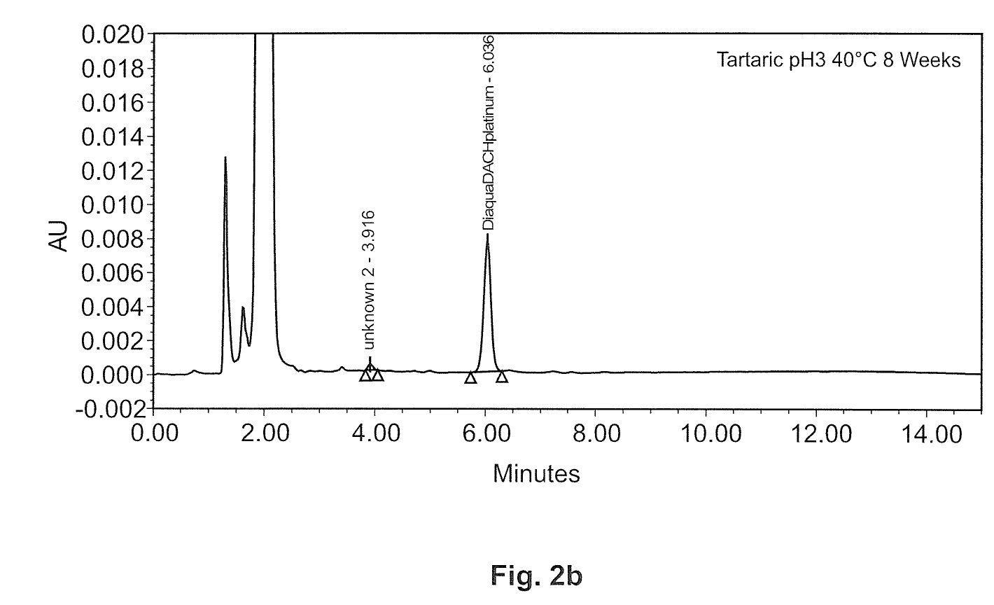

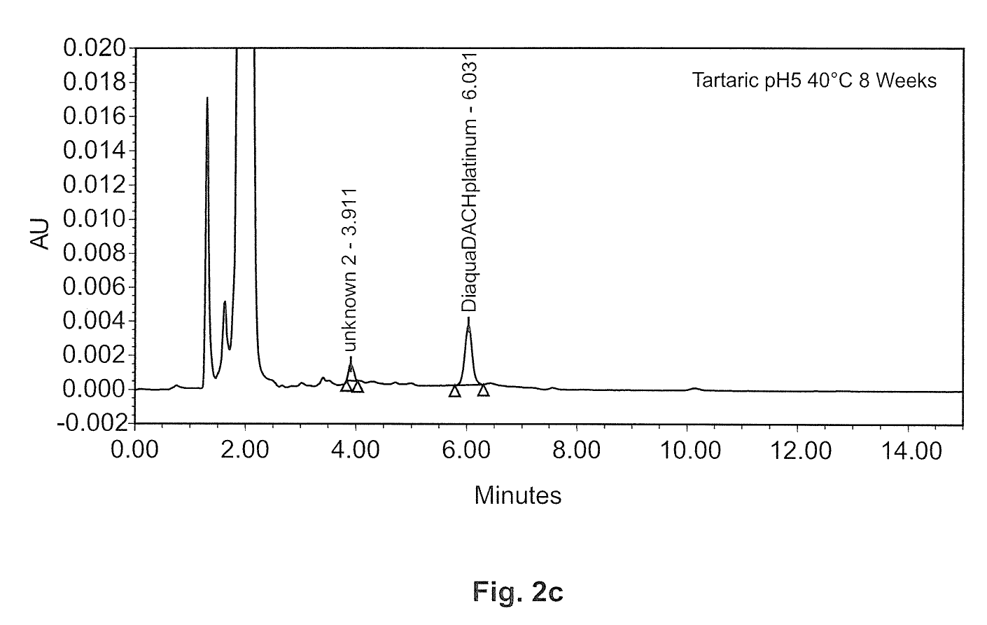

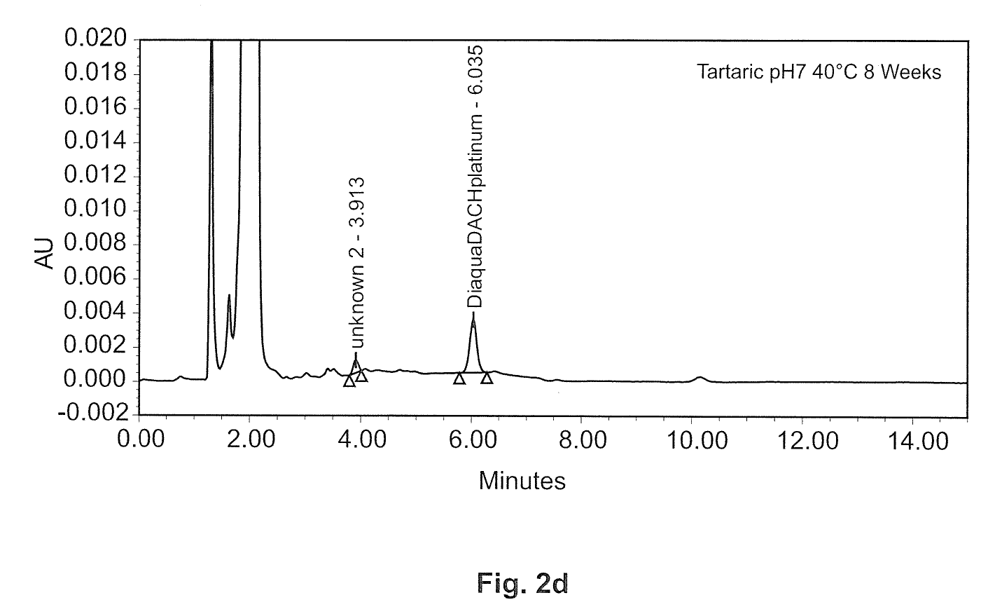

The chromatograms are presented in FIGS. 2(a)-(d)

FIG. 2(a)

### Control 40° C. 8 Weeks

This system displays an impurity peak at 6.304 minutes corresponding to Impurity B (diaqua DACH platinum) and a further peak at 10.145 minutes corresponding to Dimer (diaqua DACH platinum dimer). An unknown impurity peak is present at 3.913 minutes.

FIG. 2(b)

### Tartaric pH 3 40° C. 8 Weeks

This system displays an impurity peak at 6.306 minutes corresponding to Impurity B (diaqua DACH platinum). There is no peak corresponding to the presence of Dimer (diaqua DACH platinum dimer). An unknown impurity peak is present at 3.916 minutes.

FIG. 2(c)

### Tartaric pH 5 40° C. 8 Weeks

This system displays an impurity peak at 6.306 minutes corresponding to impurity B (diaqua DACH platinum). There is no significant peak corresponding to the presence of Dimer (diaqua DACH platinum dimer). An unknown impurity peak is present at 3.911 minutes.

FIG. 2(d)

### Tartaric pH 7 40° C. 8 Weeks

This system displays an impurity peak at 6.306 minutes corresponding to impurity B (diaqua DACH platinum). There is no significant peak corresponding to the presence of Dimer (diaqua DACH platinum dimer). An unknown impurity peak is present at 3.913 minutes.

In comparison to the chromatogram of the control formulation, Dimer (diaqua DACH platinum dimer) formation is suppressed in the tartaric acid stabilised formulations.

### Summary

In comparison to the chromatogram of the control formulation, the formation of Dimer (diaqua DACH platinum dimer) is suppressed in the tartaric acid formulations and, in some cases, significantly less Impurity B (diaqua DACH platinum), the principle degradant, is formed. Further, the tartaric acid formulations do not display as many unknown impurity peaks as the control formulation which is of importance in meeting the guide lines of the ICH and also in minimising any side effects due to the presence of unknown impurities. The increased stability of the tartaric acid formulations applies across a range of pH values.

### Example 2

### 2.1 Background

The study of this Example was conducted to further investigate the effect of different amounts of tartaric acid and the effect of pH on the stability of oxaliplatin solution formulations (Table 2). The tartaric acid formulations were compared to a control formulation of oxaliplatin in water and to a formulation of oxaliplatin in oxalic acid solution (according to U.S. Pat. No. 6,306,902).

### 2.2 Preparation of Formulations for Analysis

2.2.1 Mixing procedure for the Formulations

Add about 80% mL of desired amount of WFI into a 2 L mixing vessel and heat to 45-50° C., while stirring and flushing with nitrogen.

Add oxaliplatin (total 7.5 g) and mix until solution becomes clear.

Adjust to the volume with WFI to 1500 mL.

Divide the bulk solution to 100 mL each. Keep one 100 mL solution as the control.

Add the required amount of tartaric acid solution 5% w/v or oxalic acid and NaOH according to the formulation details in Tables 3 and 4.

Cap the final solution and keep in the refrigerator until filling.

### 2.2.2 Filling and Capping

Filter each formulation using a 0.2 μm syringe filter.

Fill 2.0 mL of each formulation filled into a 2 mL vial and cap.

Tables 3 and 4 indicate the quantities of reagents added for each different formulation.

TABLE 2
Formulation details for the oxaliplatin solutions containing tartaric acid of
Example 2

| | Item | | | | | | | | | | | | |
|---|---|---|---|---|---|---|---|---|---|---|---|---|---|
| | Tartaric | | | | | Tartaric | | Tartaric | | Tartaric | | Tartaric | |
| | (0.0003M) | | | | | (0.0006M) | | (0.0030M) | | (0.0067M) | | (0.0002M) | |
| | Formulation ID | | | | | | | | | | | | |
| | A2.5 | A4.0 | A5.0 | A7.0 | A8.5 | B4.0 | B7.0 | C4.0 | C7.0 | D4.0 | D7.0 | E4.0 | E7.0 |
| Oxaliplatin | 5 | 5 | 5 | 5 | 5 | 5 | 5 | 5 | 5 | 5 | 5 | 5 | 5 |
| (mg) | | | | | | | | | | | | | |
| Tartaric acid | 0.05 | 0.05 | 0.05 | 0.05 | 0.05 | 0.09 | 0.09 | 0.45 | 0.45 | 1.0 | 1.0 | 0.03 | 0.03 |
| (mg) | | | | | | | | | | | | | |
| WFI qs | 1 | 1 | 1 | 1 | 1 | 1 | 1 | 1 | 1 | 1 | 1 | 1 | 1 |
| (mL) | | | | | | | | | | | | | |
| Target pH | 2.5 | 4.0 | 5.5 | 7.0 | 8.5 | 4.0 | 7.0 | 4.0 | 7.0 | 4.0 | 7.0 | 4.0 | 7.0 |

Note:
Molecular weight of tartaric acid = 150.09
A = Formulation containing tartaric acid at 0.0045% (0.3 mM)
B = Formulation containing tartaric acid at 0.009% (0.6 mM)
C = Formulation containing tartaric acid at 0.045% (3 mM)
D = Formulation containing tartaric acid at 0.1% (6.7 mM)
E = Formulation containing tartaric acid at 0.003% (0.2 mM)

TABLE 3
Quantity of oxaliplatin and excipients used for the preparation of the oxaliplatin
solutions containing tartaric acid of Example 2

| | Item | | | | | | | | | | | | |
|---|---|---|---|---|---|---|---|---|---|---|---|---|---|
| | Tartaric | | | | | Tartaric | | Tartaric | | Tartaric | | Tartaric | |
| | (0.0003M) | | | | | (0.0006M) | | (0.0030M) | | (0.0067M) | | (0.0002M) | |
| | Formulation ID | | | | | | | | | | | | |
| | A2.5 | A4.0 | A5.0 | A7.0 | A8.5 | B4.0 | B7.0 | C4.0 | C7.0 | D4.0 | D7.0 | E4.0 | E7.0 |
| Oxaliplatin | 500 | 500 | 500 | 500 | 500 | 500 | 500 | 500 | 500 | 500 | 500 | 500 | 500 |
| (mg) | | | | | | | | | | | | | |
| Tartaric acid | 4.5 | 4.5 | 4.5 | 4.5 | 4.5 | 9 | 9 | 45 | 45 | 100 | 100 | 3 | 3 |
| (mg) | | | | | | | | | | | | | |
| Tartaric | 90 | 90 | 90 | 90 | 90 | 180 | 180 | 900 | 900 | 2000 | 2000 | 60 | 60 |
| acid | | | | | | | | | | | | | |
| 5% w/v | | | | | | | | | | | | | |
| (μL) | | | | | | | | | | | | | |
| WFI qs | 100 | 100 | 100 | 100 | 100 | 100 | 100 | 100 | 100 | 100 | 100 | 100 | 100 |
| (mL) | | | | | | | | | | | | | |

* The pH was adjusted to desired levels using NaOH

TABLE 4
Formulation details for the oxaliplatin control formulation and oxaliplatin
solutions containing oxalic acid of Example 2.

| | | Oxalic acid |
|---|---|---|
| Item | Control | (0.001M) |
| Oxaliplatin | 5 | 5 |
| (mg) | | |
| Oxalic acid | N/a | 0.126 |
| (mg) | | |
| WFI qs | 1 | 1 |
| (mL) | | |
| Target pH | 5.5 | 3.0 |

Note:
In the initial screening, the pH 3 tartaric formulation was formulated at pH 3.78.

### 2.3 Stability Measurements at the Initial Time Point

All of the oxaliplatin formulations at the initial time point were clear, colourless solutions with no visible particles present in solution. The appearance of the solutions are set out in Table 5. Measurements of the pH results of the formulations are also shown in Table 5.

TABLE 5
Test Results for pH and Appearance of Oxaliplatin Solutions of
Example 2 at Initial Time Point

| Formulation | pH initial | Appearance |
|---|---|---|
| Control | 5.77 | N |
| A3.5 | 3.56 | N |
| A4.0 | 3.96 | N |
| A5.0 | 5.22 | N |
| A7.0 | 7.44 | N |
| A8.5 | 8.47 | N |
| B4.0 | 4.08 | N |
| B7.0 | 7.27 | N |
| C4.0 | 3.99 | N |
| C7.0 | 6.91 | N |
| D4.0 | 3.97 | N |
| D7.0 | 7.37 | N |
| E4.0 | 3.99 | N |
| E7.0 | 7.4 | N |
| Oxalic acid | 2.94 | N |

N = a clear, colourless solution with no visible particles present in solution

### 2.4 Stability Measurements

The formulations were then stored at 25° C. and 40° C.

The appearance of the formulations was assessed at the initial, 4 week and 8 week time points. Each formulation remained clear and colourless

The pH of the formulations was measured at the initial, 4 week and 12 week time points for 25° C. as shown in Table 6 and for 40° C. as shown in Table 7.

TABLE 6
Test Results for pH of Oxaliplatin Solutions of Example 2 at 25° C.

| Formulation | pH initial | pH 4 wks | pH 12 wks |
|---|---|---|---|
| Control | 5.77 | 5.80 | 5.73 |
| A3.5 | 3.56 | 3.54 | n/t |
| A4 | 3.96 | 3.99 | 3.89 |
| A5 | 5.22 | 5.03 | 5.15 |
| A7 | 7.44 | 5.54 | 5.54 |
| A8.5 | 8.47 | 6.26 | n/t |
| B4 | 4.08 | 4.52 | n/t |
| B7 | 7.27 | 5.43 | 5.84 |
| C4 | 3.99 | 4.03 | n/t |
| C7 | 6.91 | 5.16 | 5.44 |
| D4 | 3.97 | 4.12 | n/t |
| D7 | 7.37 | 5.31 | n/t |
| E4 | 3.99 | 4.55 | 4.75 |
| E7.0 | 7.4 | 5.61 | 6.15 |
| Oxalic acid | 2.94 | 3.42 | 3.31 |

TABLE 7
Test Results for pH of Oxaliplatin Solutions of Example 2 at 40° C.

| Formulation | pH initial | pH 4 wks | pH 12 wks |
|---|---|---|---|
| Control | 5.77 | 5.79 | 3.30 |
| A3.5 | 3.56 | 3.57 | n/t |
| A4 | 3.96 | 3.93 | 3.81 |
| A5 | 5.22 | 5.06 | 5.01 |
| A7 | 7.44 | 5.43 | 5.64 |
| A8.5 | 8.47 | 6.17 | n/t |
| B4 | 4.08 | 4.29 | n/t |
| B7 | 7.27 | 5.61 | 5.95 |
| C4 | 3.99 | 4.17 | n/t |
| C7 | 6.91 | 5.25 | 5.43 |
| D4 | 3.97 | 4.14 | n/t |
| D7 | 7.37 | 5.36 | n/t |
| E4 | 3.99 | 4.24 | 4.89 |
| E7 | 7.4 | 4.23 | 6.10 |
| Oxalic acid | 2.94 | 3.25 | 3.32 |

### 2.4.1 Potency Assay

Formulations A4, A5, A7, B7, C7, E4, E7, Oxalic and the Control were maintained at 25° C. and 40° C. and were assayed for potency by HPLC after 12 weeks. Table 8 reports the impurity profile determined from the potency assay for 25° C. Table 9 reports the impurity profile determined from the potency assay for 40° C.

TABLE 8
Impurity profile from the potency assay for certain oxaliplatin formulations of
Example 2 at 12 weeks time point at 25° C.

| Impurity | Control | A4 | A5 | A7 | B7 | C7 | E4 | E7 | Oxalic |
|---|---|---|---|---|---|---|---|---|---|
| Total of | 0.10 | 0.05 | 0.05 | 0.07 | 0.08 | 0.17 | 0.10 | 0.11 | 0.15 |
| unknown | | | | | | | | | |
| impurities | | | | | | | | | |
| R,S-Oxaliplatin | 0.01 | 0.01 | 0.01 | 0.01 | 0.01 | 0.01 | 0.01 | 0.01 | 0.01 |
| Oxaliplatin | 99.87 | 99.89 | 99.89 | 99.89 | 99.88 | 99.72 | 99.86 | 99.85 | 99.76 |
| Impurity C | 0.01 | 0.01 | 0.01 | 0.01 | 0.01 | 0.01 | 0.01 | 0.01 | 0.02 |
| Total impurity (%) | 0.12 | 0.07 | 0.07 | 0.09 | 0.1 | 0.19 | 0.12 | 0.13 | 0.18 |

TABLE 9
Impurity profile from the potency assay for certain oxaliplatin formulations of
Example 2 at 12 weeks time point at 40° C.

| | | | | | | | | | Oxalic |
|---|---|---|---|---|---|---|---|---|---|
| Impurity | Control | A4.0 | A5.0 | A7.0 | B7.0 | C7.0 | E4.0 | E7.0 | acid |
| Total of | 0.15 | 0.18 | 0.13 | 0.14 | 0.08 | 0.53 | 0.07 | 0.08 | 0.15 |
| unknown | | | | | | | | | |
| impurities | | | | | | | | | |
| R,S-Oxaliplatin | 0.01 | 0.01 | 0.01 | 0.01 | 0.01 | 0.02 | 0.01 | 0.01 | 0.01 |
| Oxaliplatin | 99.67 | 99.66 | 99.74 | 99.73 | 99.81 | 99.27 | 99.77 | 99.77 | 99.66 |
| Impurity C | 0.02 | 0.02 | 0.02 | 0.01 | 0.01 | 0.02 | 0.01 | 0.01 | 0.02 |
| Total impurity (%) | 0.18 | 0.21 | 0.15 | 0.16 | 0.10 | 0.57 | 0.09 | 0.10 | 0.18 |

### 2.4.2 Impurity B Assay

The level of impurity B of the formulations maintained at 25° C. was assayed by HPLC after 12 weeks for A4, A5, A7, B7, C7, E4, E7, Oxalic and the Control. Table 10 reports the impurity profile determined from that impurity B assay for 25° C. The level of impurity B of the formulations maintained at 40° C. was assayed by HPLC after 8 weeks. Table 11 reports the impurity profile determined from that impurity B assay.

TABLE 10
The levels of impurity B and other unknown impurities from impurity B assay in
certain formulations of Example 2 at 12 weeks time point at 25° C.

| Impurity | Control | A4 | A5 | A7 | B7 | C7 | E4 | E7 | Oxalic |
|---|---|---|---|---|---|---|---|---|---|
| Total of | 0.07 | 0.05 | 0.06 | 0.06 | 0.04 | 0.06 | 0.02 | 0.06 | 0.06 |
| unknown | | | | | | | | | |
| impurity | | | | | | | | | |
| Imp B | 0.24 | 0.29 | 0.12 | 0.10 | 0.13 | 0.06 | 0.21 | 0.18 | 0.38 |
| Dimer | 0.10 | Nd | Nd | Nd | 0.02 | Nd | Nd | 0.05 | Nd |
| Total impurity (%) | 0.41 | 0.34 | 0.18 | 0.16 | 0.19 | 0.12 | 0.23 | 0.29 | 0.44 |

TABLE 11
The levels of impurity B and other unknown impurities from impurity B assay in
certain formulations of example 2 at the 8 weeks time point at 40° C.

| Impurity | Control | A4 | A5 | A7 | B7 | C7 | E4 | E7 | Oxalic |
|---|---|---|---|---|---|---|---|---|---|
| Total of unknown | 0.15 | 0.06 | 0.09 | 0.10 | 0.09 | 0.11 | 0.06 | 0.07 | 0.05 |
| impurities | | | | | | | | | |
| Imp B | 0.26 | 0.24 | 0.09 | 0.09 | 0.11 | 0.06 | 0.19 | 0.19 | 0.36 |
| Dimer | 0.17 | 0.01 | Nd | Nd | 0.03 | 0.01 | Nd | Nd | Nd |
| Total impurity (%) | 0.58 | 0.31 | 0.18 | 0.19 | 0.23 | 0.18 | 0.25 | 0.26 | 0.41 |

### 2.5 Stability Measurements at 9 Months

Formulations Control, A4.0, A5.0, A7.0, E4.0 and E7.0 were stored at 25° C. and 40° C. for 9 months and then analysed for pH and impurities.

### 2.5.1 Results and Discussion

### 2.5.1.1 Appearance Results

The appearance of the formulations Control, A4.0, A5.0, A7.0, E4.0 and E7.0 was clear and colourless after being stored at 25° C. and 40° C. for 9 months.

### 2.5.1.2 Impurity B Assay

Levels of impurity B and Dimer in formulations Control, A4, A5, E4 and E7 at 9 months for both 25° C. and 40° C. were assessed using HPLC. The results are shown in Table 12 and 13, respectively.

From the assay, the formulations A4, A5, E4 and E7 contained less total impurity than control at 25° C. At 40° C., formulations A4, A5 and E4 contained less total impurity than the control. In all cases the Dimer impurity was suppressed relative to the Control and indeed was not detected in formulations A5, A7 and E4.

TABLE 12
The % of impurity B and other unknown impurities from impurity B
assay in certain formulations of Example 2 at 25° C. for 9 months

| Impurities | Control | A4 | A5 | E4 | E7 |
|---|---|---|---|---|---|
| Total of | 0.07 | 0.05 | 0.06 | 0.03 | 0.10 |
| unknown | | | | | |
| impurities | | | | | |
| Imp B | 0.22 | 0.27 | 0.11 | 0.18 | 0.15 |
| dimer | 0.15 | ND | ND | ND | 0.06 |
| Total impurity | 0.44 | 0.32 | 0.17 | 0.21 | 0.31 |

ND = not detected.

TABLE 13
The % of impurity B and other unknown impurities from
impurity B assay in certain formulations of Example 2
at 9 months time point at 40° C.

| Impurities | Control | A4 | A5 | A7 | E4 | E7 |
|---|---|---|---|---|---|---|
| Total of unknown | 0.22 | 0.20 | 0.31 | 0.42 | 0.22 | 0.46 |
| impurities | | | | | | |
| Imp B | 0.26 | 0.21 | 0.09 | 0.08 | 0.23 | 0.12 |
| dimer | 0.14 | ND | ND | ND | ND | 0.04 |
| Total impurity | 0.62 | 0.41 | 0.40 | 0.50 | 0.45 | 0.62 |

ND = not detected

### 2.6 Summary

The screening study indicated that tartaric acid is suitable for use with oxaliplatin at a range of concentrations. In terms of the ability of tartaric acid to stabilise the oxaliplatin, concentrations of 0.2 mM and 0.3 mM (formulations E and A respectively) are preferred, although formulations at 0.6 mM (formulations B) also demonstrated some stabilising effect.

### Example 3

The following formulation was prepared for the purpose of regulatory testing:

Oxaliplatin 5 mg

Tartaric acid 0.03 mg

NaOH (adjust to pH of approximately 5)

WFI qs 1 mL

The pH is adjusted to pH 5 with a range of from 4.7 to 5.5 using NaOH. The concentration of tartaric acid is about 0.2 mM.

Throughout this specification, the word “comprise”, or variations such as “comprises” or “comprising” will be understood to imply the inclusion of a stated element, integer or step, or groups of elements, integers or steps, but not the exclusion of any other element, integer or step, or groups of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of the application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

We claim:

1. A pharmaceutical liquid formulation of oxaliplatin for parenteral administration, said formulation comprising
(i) oxaliplatin,
(ii) water; and
(iii) an additive selected from the group consisting of tartaric acid; a salt of tartaric acid; a pharmaceutically acceptable derivative of tartaric acid wherein said pharmaceutically acceptable derivative is selected from the group consisting of esters, amides, carbonates and carbamates of tartaric acid; and mixtures thereof.

2. A formulation according to claim 1, wherein the concentration of the additive is from 0.01 mM to about 2.0 mM.

3. A formulation according to claim 1, wherein the concentration of the additive is from 0.1 mM to about 1.0 mM.

4. A formulation according to claim 1, wherein the concentration of the additive is from 0.1 mM to about 0.6 mM.

5. A formulation according to claim 1, wherein the concentration of the additive is from 0.2 mM to about 0.6 mM.

6. A formulation according to claim 1, wherein the additive comprises sodium tartrate.

7. A formulation according to claim 1, wherein the concentration of oxaliplatin is up to about 15 mg/ml.

8. A formulation according to claim 1, wherein the concentration of oxaliplatin is up to about 7 mg/ml.

9. A formulation according to claim 1, wherein the pH of the formulation is in the range of from 3 to about 7.

10. A method for treating a cancer which comprises administering a pharmaceutical formulation according to claim 1 to a patient in need thereof.

11. A method for preparing a pharmaceutical formulation, the method comprising the steps of:
(i) dissolving oxaliplatin in water to form a solution;
(ii) dissolving in the solution an additive selected from the group consisting of a tartaric acid; a salt of tartaric acid; a pharmaceutically acceptable derivative of a pharmaceutically acceptable tartaric acid wherein said pharmaceutically acceptable derivative is selected from the group consisting of esters, amides, carbonates and carbamates of tartaric acid; and mixtures thereof;
(iii) optionally, adjusting the pH of the solution with a pharmaceutically acceptable base to form said pharmaceutical formulation.

12. A method according to claim 11, wherein the concentration of the additive is from 0.01 mM to about 2.0 mM.

13. A method according to claim 11, wherein the concentration of the additive is from 0.1 mM to about 1.0 mM.

14. A method according to claim 11, wherein the concentration of the additive is from 0.1 mM to about 0.6 mM.

15. A method according to claim 11, wherein the concentration of the additive is from 0.2 mM to about 0.6 mM.

16. A method according to claim 11, wherein the additive comprises a salt of tartaric acid and wherein the salt is a sodium salt.

17. A method according to claim 11, wherein the concentration of oxaliplatin is up to about 15 mg/ml.

18. A method according to claim 11, wherein the concentration of oxaliplatin is up to about 7 mg/ml.

19. A method according to claim 11, wherein the concentration of oxaliplatin is about 5 mg/ml.

20. A method according to claim 11, wherein the pharmaceutically acceptable base is sodium hydroxide.

21. A method according to claim 11, wherein the pH of the formulation is adjusted to be in the range of from 3 to 7.

22. A pharmaceutical liquid formulation of oxaliplatin for parenteral administration, said formulation comprising
(i) about 5 mg/ml of oxaliplatin,
(ii) water, and
(iii) an additive consisting of tartaric acid and the sodium salt of tartaric acid,
wherein the concentration of the additive is about 0.2 mM and wherein the pH of the solution is from 4.7 to 5.5.

23. A method for treating a cancer which comprises administering a pharmaceutical formulation according to claim 22 to a patient in need thereof.

24. A method for preparing a pharmaceutical formulation, the method comprising the steps of:
(i) dissolving oxaliplatin in water to form a solution;
(ii) dissolving tartaric acid in the solution;
(iii) adjusting the pH of the solution with sodium hydroxide such that it is in the range of from 4.7 to 5.5 to form said pharmaceutical formulation
wherein the concentration of oxaliplatin is about 5 mg/ml and the concentration of tartaric acid is about 0.2 mM.
